(19) United States
(12) Patent Application Publication
(10) Pub. No.: US 2022/0387667 A1
(43) Pub. Date: Dec. 8, 2022

(54) METHODS OF PRODUCING DECELLULARIZED AND RECELLULARIZED PROSTATE TISSUES AND TREATING BENIGN PROSTATIC HYPERPLASIA OR PROSTATE CANCER

(71) Applicant: AtRoo, Inc., Watertown, MA (US)

(72) Inventors: Abdol-Mohammad KAJBAFZADEH, Tehran (IR); Aram AKBARZADEH, Cambridge, MA (US)

(73) Assignee: AtRoo, Inc., Watertown, MA (US)

(21) Appl. No.: 17/703,316

(22) Filed: Mar. 24, 2022

Related U.S. Application Data
(60) Provisional application No. 63/165,959, filed on Mar. 25, 2021.

(51) Int. Cl. A61L 27/36; A61K 9/00; C12N 5/071; A61K 35/52

(52) U.S. Cl. CPC A61L 27/3604; A61K 9/0024; C12N 5/0683; A61K 35/52; A61L 27/3687; C12N 2533/90; A61L 2430/40

(57) ABSTRACT

The subject matter described herein is directed to methods of producing decellularized and recellularized human prostate tissues and methods of treating disorders of a prostate, including BPH and prostate cancer, are disclosed. Prostate tissue engineering and the composition and construction of a prostate extracellular matrix (ECM) or a prostate tissue scaffold and interactions with its cellular components are also disclosed.

### CROSS REFERENCE TO RELATED APPLICATIONS

This application claims the benefit of U.S. Provisional patent application 63/165,959 filed Mar. 25, 2021, which is hereby incorporated by reference.

### FIELD

Some aspects of this disclosure provide methods of producing decellularized and recellularized human prostate tissues and methods of treating disorders of a prostate, including BPH and prostate cancer. Prostate tissue engineering and the composition and construction of a prostate extracellular matrix (ECM) or a prostate tissue scaffold and interactions with its cellular components are also disclosed.

### BACKGROUND

Prostate cancer is in nature a biologically heterogeneous disease with complicated origin which affords noteworthy confronts for its clinical administration.

Prostate cancer is an increasing global concern and the second commonest male cancer globally. Based on GLOBOCAN 2018 estimates, 1,276,106 new cases of prostate cancer and 358,989 deaths were reported worldwide in 2018. The clinical features of the disease include a wide spectrum, from low-grade to aggressive high-grade end-stage malignancies that could lead to metastases and death (Antonarakis E S, Shaukat F. Velho P I, Kaur H. Shenderov E, Pardoll D M, Lotan T L (2019) Clinical features and therapeutic outcomes in men with advanced prostate cancer and DNA mismatch repair gene mutations. European urology 75 (3):378-382). Significantly, 10-33% of clinically localized cancers will progress following surgery, which demonstrates undetected pre-existing metastatic disease (AMLING C L, Blute M L, Bergstralh E J, Seay T M, Slezak J, Zincke H (2000) Long-term hazard of progression after radical prostatectomy for clinically localized prostate cancer: continued risk of biochemical failure after 5 years. The Journal of urology 164 (1):101-105).

Although Gleason grading system pathology is able to score epithelial differentiation in prognosis and management; it is imprecise in managing low grade prostate tumors which are at risk of rapid progression (Epstein J I, Egevad L, Amin M B, Delahunt B, Srigley J R, Humphrey P A (2016) The 2014 International Society of Urological Pathology (ISUP) consensus conference on Gleason grading of prostatic carcinoma. The American journal of surgical pathology 40 (2):244-252).

Several interventions for men with localized prostate cancer comprise active surveillance, adjuvant chemotherapy, hormonal therapy, surgery, or radiotherapy with curative intent; however, these therapeutic options have also various complications (Tosco L, Briganti A, D'anico A V, Eastham J, Eisenberger M, Gleave M, Haustermans K, Logothetis C J, Saad F, Sweeney C (2019) Systematic review of systemic therapies and therapeutic combinations with local treatments for high-risk localized prostate cancer. European urology 75 (1):44-60).

Due to the nature characterization of prostate cancer as being interpatient, (multifocal disease), intratumorally, genomic, and epigenetic heterogeneity, development of treatment options such as, radiation chemotherapy, surgical and more, raises considerable challenges. Nevertheless, tissue-engineering and regenerative medicine techniques for cancer research have been recently emerged as a probable method for the study of prostate metastasis. Bidirectional signaling between cells and the extracellular matrix (ECM) proteins (collagen, proteoglycans, laminin, and fibronectin) play a fundamental role in prostate development and homeostasis (Leach D A, Need E F, Toivanen R, Trotta A P, Palenthorpe H M, Tamblyn D J, Kopsaftis T, England G M, Smith E, Drew P A (2015) Stromal androgen receptor regulates the composition of the microenvironment to influence prostate cancer outcome. Oncotarget 6 (18):16135). The composition of the ECM (fibrillar protein type I V collagen) and this bidirectional signaling is altered in prostate cancer (Clark A K, Taubenberger A V, Taylor R A, Niranjan B, Chea Z Y, Zotenko E, Sieh S, Pedersen J S, Norden S, Frydenberg M (2013) A bioengineered microenvironment to quantitatively measure the tumorigenic properties of cancer-associated fibroblasts in human prostate cancer. Biomaterials 34 (20):4777-4785). The use of prostate ECM, scaffolds may have an impact on the development of an innovative personalized tissue engineering therapy as substitute and efficient treatments for several prostate diseases.

Aging as the greatest risk factors for both benign and malignant pathology, results in interactions between components of the ECM and prostate epithelial cells. In addition, type of the ECM may noticeably influence experimental and clinical treatment outcomes. It has been also demonstrated that cell seeding in different types of ECM culminated in ECM-induced cell-type dependent modifications in cell differentiation and morphogenesis (Čunderliková B, Filová B, Kajo K, Vallová M, Balázsiová Z, Trnka M. Mateaik A (2018) Extracellular matrix affects different aspects of cell behaviour potentially involved in response to aminolevulinic acid-based photoinactivation. Journal of Photochemistry and Photobiology B: Biology 189:283-291).

Understanding the basis of cancer cell-ECM interactions is critical to increase the perception of tumor progression, and also offers opportunities to establish more effective therapeutic strategies.

Benign prostatic hyperplasia (BPH) is common in older men, with symptoms that impact quality of life, including interference with activities and perception of well-being. BPH can be progressive, with risk of urinary retention, infections, bladder calculi and renal failure. Although many men with mild to moderate symptoms do well without intervention, bothersome symptoms and complications can progress in others, leading to medical therapy or surgery.

BPH is caused by increased activity of both androgens and estrogens. Because of such a dual etiology of BPH, proposed hormonal therapies have been less than satisfactory and have all been unpredictable while, frequently, causing unacceptable side-effects. Moreover, the prior art treatments seldomly resulted in a decrease in prostatic volume above about 20 to 30% with inconsistent effects on the symptomatology (Scott and Wade, J. Urol. 101: 81-85, 1969; Caine et al., J. Urol. 114: 564-568, 1975; Peters and Walsh, New Engl. J. Med. 317: 599-604, 1987: Gabrilove et al., J. Clin. Endocrinol. Metab. 64: 1331-1333, 1987; Stone et al., J. Urol. 141: 240A, 1989; Clejan et al., J. Urol. 141: 534A, 1989; Stoner, E., Lecture on the role of 5α-reductase inhibitor in benign prostatic hypertrophy, 84th AUA Annual Meeting, Dallas, May 8, 1989).

Benign tumors and malformations can be treated by a variety of methods including surgery, radiotherapy, drug therapy, thermal or electric ablation, cryotherapy, and others. Although benign tumors do not metastasize, they can grow large and they can recur. Surgical extirpation of benign tumors has all the difficulties and side effects of surgery in general and oftentimes must be repeatedly performed for some benign tumors, such as for pituitary adenomas, meningeomas of the brain, prostatic hyperplasia, and others. In addition, some patients who receive non-surgical treatment to ameliorate the symptoms caused by benign tumors, still require subsequent invasive surgical intervention. Lepor, “Medical Treatment of Benign Prostatic Hyperplasia,” Reviews in Urology. Vol. 13, No. 1, pp. 20-33 (2011), discloses a variety of studies of the efficacy of drug therapies in treating BPH, and the need for subsequent invasive surgical treatment due to worsening or progression of symptoms of BPH.

Various 3D systems have been examined to reproduce the tumor microenvironment in-vitro. Most of these systems are either synthetic or derived from animals.

Behavior of the ECM and its influence on tumor development and therapeutic efficacy is still one of the unanswered questions yet needs to be addressed. The type of the ECM has been identified as playing an essential role in driving cancer progression which may obviously influence experimental and clinical treatment results.

The constituents of the ECM can be degraded and modified during a remodeling process. A tissue-specific microenvironment will shape by the arrangement and direction of ECM components which plays a key role in tumor progression. This application newly discloses the correlation between P63 expression and tumor cell proliferation (Ki-67) and apoptosis (TUNEL staining). Increased levels of Alfa-methyl-acyl-CoA racemase (AMACR) marker and lack of P63 is believed to enhance the risk of emerging prostate cancer, with increased tumor progression and a poor patient prognosis. Disclosed herein are ECM decellularization and recellularization methods for prostate treatment and replacement.

The present invention is based, in part, on the discovery that cell-ECM interactions, and the expression of extracellular matrix components in the prostate tissue engineering, can be used for further cancer therapy in patients.

The present invention provides results regarding decellularization and recellularization of natural, benign prostatic hyperplasia (BPH), and malignant human prostatic tissues, and confirms the role of extracellular matrix behavior on development of prostate cancer.

Various ECM proteins such as integrins, collagen, fibronectin, and vimentin engage in cell adhesion, invasion and metastasis, which are decisive for prostate cancer progression.

In-vivo cell seeding of different types of ECM in rat model culminates in ECM-induced cell-type dependent modifications in cell differentiation and morphogenesis.

The expression of some markers may change in malignant human prostatic tissues after decellularization process.

The present invention overcomes the problems and disadvantages associated with current strategies for prostate cancer treatment.

### SUMMARY

In some embodiments, the present teachings provide methods of producing a decellularized tissue or organ scaffold comprising: a) providing a tissue or organ sample, and b) treating said tissue or organ sample, thereby producing a decellularized tissue or organ scaffold.

In some embodiments, the present teachings provide methods of producing a decellularized prostate tissue scaffold comprising: (i) rinsing a prostate tissue sample with 1% Triton X-100; (ii) washing said sample with phosphate buffered saline (PBS); and (iii) preserving said sample in 1% sodium dodecyl sulfate (SDS); thereby producing a decellularized prostate tissue scaffold.

In some embodiments, the present teachings provide methods of producing a decellularized prostate tissue scaffold comprising: (i) rinsing a prostate tissue sample with 1% Triton X-100; (ii) washing said sample with phosphate buffered saline (PBS); (iii) preserving said sample in 1% sodium dodecyl sulfate (SDS); (iv) preserving said sample in 0.05% Trypsin solution; (v) rinsing said sample with PBS; (vi) using 2% SDS solution on said sample; and (vii) washing said sample with PBS; thereby producing a decellularized prostate tissue scaffold

In some embodiments, the present teachings provide methods of producing a decellularized prostate tissue scaffold comprising: (i) rinsing a prostate tissue sample with 1% Triton X-100 at 37° C. for 24 hours; (ii) washing said sample with phosphate buffered saline (PBS) at 4° C. for 24 hours; (iii) preserving said sample in 1% sodium dodecyl sulfate (SDS) at 37° C. for 48 hours; (iv) preserving said sample in 0.05% Trypsin solution on a shaker at 37° C. for 30 minutes; (v) rinsing said sample with PBS at 4° C. for 48 hours; (vi) using 2% SDS solution on said sample at 37° C. for 48 hours; and (vii) washing said sample with PBS at 4° C. for 48 hours; thereby producing a decellularized prostate tissue scaffold.

In some embodiments, the methods taught herein further comprise, following step (vii): (viii) preserving the decellularized prostate tissue scaffold in an antibiotic solution at 4° C.

In some embodiments, the decellularized prostate tissue scaffolds taught herein are repopulated with cells to produce a recellularized prostate tissue scaffold.

In some embodiments, the recellularization is done by in-vivo implantation of the decellularized prostate tissue scaffold in a human subject.

In some embodiments, following recellularization of the prostate tissue scaffold, the recellularized prostate tissue does not contain cancerous cells.

In some embodiments, the present teachings provide a decellularized prostate tissue scaffold produced by the methods described herein.

In some embodiments, the present teachings provide an extracellular matrix produced by the methods described herein.

In some embodiments, the present teachings provide recellularized prostate tissue scaffold produced by the methods described herein.

In some embodiments, the present teachings provide methods of treating a disorder of the prostate by implanting the decellularized prostate tissue scaffolds described herein.

In some embodiments, the disorder of the prostate being treated is benign prostatic hyperplasia (BPH).

In some embodiments, the disorder of the prostate being treated is prostate cancer.

Other advantages, features, and characteristics of the present invention will become more apparent upon consideration of the following detailed description and claims.

### BRIEF DESCRIPTION OF THE DRAWINGS

The following drawings form part of the present specification and are included to further demonstrate certain aspects of the present disclosure, the inventions of which can be better understood by reference to one or more of these drawings in combination with the detailed description of specific embodiments presented herein.

FIGS. 1A-1H: FIG. 1A depicts Normal prostate. FIG. 1B shows a decellularized prostate scaffold. FIGS. 1C-1F shows the Surgical technique. FIGS. 1G-1H show a biopsy after 6 months of follow-up.

FIGS. 2A-2B: FIG. 2A shows the results of the DNA quantification before and after the decellularization process. FIG. 2B shows the results of the MTT assay in the provided bio-scaffolds.

FIGS. 3A-3B: FIG. 3A depicts H&E, DAPI, and Trichrome staining in normal and decellularized normal prostate. FIG. 3B shows Fibronectin, Vimentin, and Laminin staining in normal and decellularized normal prostate.

FIGS. 4A-4B: FIG. 4A shows H&E, DAPI, and Trichrome staining in BPH and decellularized BPH. FIG. 4B shows Fibronectin, Vimentin, and Laminin staining in BPH and decellularized BPH.

FIGS. 5A-5B: FIG. 5A shows H&E, DAPI, and Trichrome staining in normal prostate cancer tissue and decellularized prostate cancer tissue. FIG. 5B shows Fibronectin, Vimentin, and Laminin staining in normal prostate cancer tissue and decellularized prostate cancer tissue.

FIGS. 6A-6C show Collagen Type I (FIG. 6A), Collagen Type III (FIG. 6B), and Collagen Type IV (FIG. 6C) staining in BPH, decellularized BPH, normal prostate cancer tissue, and decellularized prostate cancer tissue.

FIG. 7 shows TUNEL staining before and after the decellulanzation process (A-F), and fluorescent microscopy of normal, BPH, and malignant tissue with the presence of GFP-positive cells in in the recellularized scaffolds (G-I).

FIG. 8 shows histopathological evaluations of implanted normal prostate scaffold after 6 months of follow-up.

FIG. 9 depicts histopathological evaluations of implanted decellularized BPH after 6 months of follow-up.

FIG. 10 depicts histopathological evaluations of implanted decellularized prostate cancer tissue after 6 months of follow-up.

### DETAILED DESCRIPTION

In order to have a clearer and more consistent understanding of the report and the claims, including the scope given to said terms, the following definitions are provided.

Terms and phrases used herein are defined as set forth below unless otherwise specified. Throughout this description, the singular forms “a,” “an,” and “the” include plural reference unless the context clearly dictates otherwise. Thus, for example, a reference to “a cell” includes a plurality of such cells, and a reference to “an antibiotic” is a reference to one or more antibiotics and equivalents thereof known to those skilled in the art, and so forth.

The term “extracellular matrix”, abbreviated “ECM”, refers to the complex structural material that is produced by cells in mammalian tissues, particularly cells of connective tissue, for instance such cells as fibroblasts, osteoblasts, chondrocytes, epithelial cells, smooth muscle cells, adipocytes, and mesenchymal cells, and which material in vivo surrounds and supports those cells. Typically, the ECM is composed of fibers embedded in what is commonly referred to as “ground substance”. The fibers are composed of structural proteins, generally collagen and/or elastin. In aspects of the present invention, the fibers of the matrix are preferably collagen.

The term “matrix” refers to the structural component of the cell microenvironment. Matrix is also commonly referred to as “extracellular matrix” or “ECM.”

The term “human tissue sample” refers to a “human organ sample” or a “human prostate sample”. Similarly, the term “tissue sample” refers to an “organ sample” or a “prostate sample”.

A male with worsening or progression of symptoms of BPH would be understood by a person having ordinary skill in the art to be a mammal having an increase in IPSS score over time, or showing no improvement (i.e., reduction) of IPSS over time. A person having ordinary skill in the art knows and understands how to determine an IPSS score, using well-known and industry-accepted standards.

The International Prostate Symptom Score (I-PSS) is based on the answers to seven questions concerning urinary symptoms and one question concerning quality of life. Each question concerning urinary symptoms allows the patient to choose one out of six answers indicating increasing severity of the particular symptom. The answers are assigned points from 0 to 5. The total score can therefore range from 0 to 35 (asymptomatic to very symptomatic). The first seven questions of the IPSS are identical to the questions appearing on the American Urological Association (AUA) Symptom Index.

The International Scientific Committee (SCI), under the patronage of the World Health Organization (WHO) and the International Union Against Cancer (UICC), recommends the use of only a single question to assess the quality of life. The answers to this question range from “delighted” to “terrible” or 0 to 6. Although this single question may or may not capture the global impact of benign prostatic hyperplasia (BPH) Symptoms or quality of life, it may serve as a valuable starting point for a doctor-patient conversation.

Not all males suffering from BPH exhibit worsening or progression of symptoms associated with BPH. In other words, some males suffering from BPH may not see an increase in their IPSS score over time, even though they continue to suffer from the disorder. Similarly, some males suffering from BPH may see a dramatic increase in IPSS score over time. The embodiments described herein may include treating mammals suffering from BPH that are susceptible to worsening or progression of symptoms of BPH, as evinced by an increase in IPSS over time, or by the lack of any appreciable reduction in IPSS over time. In another embodiment, the males treated are those that have or are susceptible to having BPH.

The embodiments include a method of improving sexual function in a male with BPH comprising recellularizing prostate tissue scaffolds in a human male.

Prostate cancer is a disease in which cancer develops in the prostate, a gland in the male reproductive system. It occurs when cells of the prostate mutate and begin to multiply out of control. Typical antigens which have been shown to be overexpressed by prostate cancer cells as compared to normal counterparts are inter alia antigens like PSA, PSMA, PAP, PSCA, HER-2 and Ep-CAM. These prostate cancer cells may spread (metastasize) from the prostate to other parts of the body, especially the bones and lymph nodes. Prostate cancer may cause pain, difficulty in urinating, erectile dysfunction and other symptoms. Typically, prostate cancer develops most frequently in men over fifty, which represent the most common group of patients. However, prostate cancer remains most often undiscovered, even if determination would be possible. Determination of prostate cancer typically occurs by physical examination or by screening blood tests, such as the PSA (prostate specific antigen) test. When suspected to prostate cancer the cancer is typically confirmed by removing a piece of the prostate (biopsy) and examining it under a microscope. Further tests, such as X-rays and bone scans, may be performed to determine whether prostate cancer has spread.

Treatment of prostate cancer still remains an unsolved challenge. Conventional therapy methods may be applied for treatment of prostate cancer such as surgery, radiation therapy, hormonal therapy, occasionally chemotherapy, proton therapy, or some combination of these. However, the age and underlying health of the man as well as the extent of spread, appearance under the microscope, and response of the cancer to initial treatment are important in determining the outcome of the disease. Since prostate cancer is a disease, typically diagnosed in older men, many will die of other causes before a slowly advancing prostate cancer can spread or cause symptoms. This makes treatment selection difficult. The decision whether or not to treat localized prostate cancer (a tumor that is contained within the prostate) with curative intent is a trade-off between the expected beneficial and harmful effects in terms of patient survival and quality of life.

However, the above therapy methods, such as surgery, radiation therapy, hormonal therapy, and chemotherapy, etc., all suffer from severe limitations. By way of example, surgical removal of the prostate, or prostatectomy, is a common treatment either for early stage prostate cancer or for cancer which has failed to respond to radiation therapy. It may cause nerve damage that significantly alters the quality of life. The most common serious complications are loss of urinary control and impotence. However, even if the prostate cancer could be removed successfully, spread of prostate cancer throughout the organism remains an unsolved problem.

Radiation therapy is commonly used in prostate cancer treatment. It may be used instead of surgery for early cancers, and it may also be used in advanced stages of prostate cancer to treat painful bone metastases. Radiation treatments also can be combined with hormonal therapy for intermediate risk disease, when radiation therapy alone is less likely to cure the cancer. However, radiation therapy also bears high risks and often leads to a complete loss of immune defense due to destruction of the patient's immune system. Furthermore, radiation therapy is typically applied locally at the site of cancer growth and thus may not avoid the above problem of spread of prostate cancer throughout the organism. If applied systemically, radiation therapy may lead to severe damages to cells and immune system.

Chemotherapy was considered as a less effective sort of treatment for prostate cancers since only very few patients even respond to this sort of therapy. However, some patients (responders), having a metastasizing prostate carcinoma, may benefit from chemotherapy. The response rate is at about 20% and chemotherapy will thus play a role during treatment of the tumor relapse and failing of hormonal therapy. However, chemotherapy will typically be only palliative and does not lead to a total elimination of the prostate cancer in the patient. Typical chemotherapeutic agents include cyclophosphamid, doxorubicin, 5-fluoruracil, adriamycin, suramin and other agents, however, none of these resulted in a significant longer survival of the patients. In a more recent study, published 2004 in the New England Journal of Medicine, longer survival of median 2.5 months could be demonstrated for patients which received a dosis of the agent docetaxel every three weeks (Tannock I F et al. Docetaxel plus prednisone or mitoxantrone plus prednisone for advanced prostate cancer. N Engl J Med. 2004 Oct. 7; 351(15):1502-12).

Hormonal therapy typically uses medications or a combination of hormonal therapy with surgery to block prostate cancer cells from getting dihydrotestosterone (DHT), a hormone produced in the prostate and required for the growth and spread of most prostate cancer cells. Blocking DHT often causes prostate cancer to stop growing and even shrink. However, hormonal therapy rarely cures prostate cancer because cancers which initially respond to hormonal therapy typically become resistant after one to two years. E.g. palliative androgen deprivation therapy can induce remissions in up to 80% of the patients, but after 15-20 months, tumor cells become hormone-insensitive and androgen-independent prostate cancer develops. In this situation treatment options are rare, as chemotherapy has been of limited efficacy (see above). Hormonal therapy is therefore usually used when cancer has spread from the prostate. It may also be given to certain men undergoing radiation therapy or surgery to help prevent return of their cancer.

In one embodiment, disclosed herein is a method of producing a decellularized tissue scaffold comprising: a) providing a tissue sample, and a) treating said tissue sample, thereby producing a decellularized tissue scaffold.

In one embodiment, disclosed herein is a method of producing a decellularized tissue scaffold comprising a) providing a tissue sample, and b) treating the tissue sample with a detergent, thereby producing a decellularized tissue scaffold. In another embodiment, disclosed herein is a method of producing a decellularized human tissue scaffold comprising a) providing a human tissue sample, and b) treating the human tissue sample with a detergent, thereby producing a decellularized human tissue scaffold. In another embodiment, disclosed herein is a method of producing a decellularized prostate tissue scaffold comprising a) providing a prostate tissue sample, and b) treating said prostate tissue sample with a detergent, thereby producing a decellularized prostate tissue scaffold.

In one embodiment, disclosed herein is a method of producing a decellularized tissue scaffold comprising a) providing a tissue sample, and b) treating the tissue sample with a detergent under continuous shaking, thereby producing a decellularized tissue scaffold. In another embodiment, disclosed herein is a method of producing a decellularized human tissue scaffold comprising a) providing a human tissue sample, and b) treating the human tissue sample with a detergent under continuous shaking, thereby producing a decellularized human tissue scaffold. In another embodiment, disclosed herein is a method of producing a decellularized prostate tissue scaffold comprising a) providing a prostate tissue sample, and b) treating said prostate tissue sample with a detergent under continuous shaking, thereby producing a decellularized prostate tissue scaffold.

In one embodiment, disclosed herein is a method of producing a decellularized organ scaffold comprising: a) providing an organ sample, and b) treating said organ sample, thereby producing a decellularized organ scaffold.

In one embodiment, disclosed herein is a method of producing a decellularized organ scaffold comprising a) providing an organ sample, and b) treating the organ sample with a detergent, thereby producing a decellularized organ scaffold. In another embodiment, disclosed herein is a method of producing a decellularized human organ scaffold comprising a) providing a human organ sample, and b) treating the human organ sample with a detergent, thereby producing a decellularized human organ scaffold. In another embodiment, disclosed herein is a method of producing a decellularized prostate organ scaffold comprising a) providing a prostate organ sample, and b) treating said prostate organ sample with a detergent, thereby producing a decellularized prostate organ scaffold.

In one embodiment, disclosed herein is a method of producing a decellularized organ scaffold comprising a) providing an organ sample, and b) treating the organ sample with a detergent under continuous shaking, thereby producing a decellularized organ scaffold. In another embodiment, disclosed herein is a method of producing a decellularized human organ scaffold comprising a) providing a human organ sample, and b) treating the human organ sample with a detergent under continuous shaking, thereby producing a decellularized human organ scaffold. In another embodiment, disclosed herein is a method of producing a decellularized prostate organ scaffold comprising a) providing a prostate organ sample, and b) treating said prostate organ sample with a detergent under continuous shaking, thereby producing a decellularized prostate organ scaffold.

In one embodiment, disclosed herein is a method of producing a decellularized tissue scaffold comprising (i) rinsing a tissue sample with cation detergent, for example 1% Triton X-100; (ii) washing said sample with phosphate buffered saline (PBS); (iii) preserving said sample in 1% sodium dodecyl sulfate (SDS). In another embodiment, the steps (i) through (iii) are performed sequentially.

In one embodiment, the methods of producing a decellularized tissue scaffold disclosed herein further comprise, after step (iii), the steps of: (iv) preserving said sample in 0.05% Trypsin solution; and (v) rinsing said sample with PBS. In another embodiment, the steps (i) through (v) are performed sequentially.

In one embodiment, the methods of producing a decellularized tissue scaffold disclosed herein further comprise, after step (v), the steps of: (vi) using 2% SDS solution on said sample; and (vii) washing said sample with PBS. In another embodiment, the steps (i) through (vii) are performed sequentially.

In one embodiment, the methods of producing a decellularized tissue scaffold disclosed herein further comprise, after step (vii), the step of: (viii) preserving the decellularized prostate tissue scaffold in an antibiotic solution. In another embodiment, the steps (i) through (viii) are performed sequentially.

In one embodiment, step (i) of the methods of producing a decellularized tissue scaffold disclosed herein is performed at 37° C. for 24 hours. In another embodiment, step (i) of the methods of producing a decellularized tissue scaffold is performed at about 37° C. for about 24 hours. In another embodiment, step (i) of the methods of producing a decellularized tissue scaffold is performed at about between 20° C. to 51° C. In another embodiment, step (i) of the methods of producing a decellularized tissue scaffold is performed at about between 12 hours to 36 hours. In another embodiment, step (i) of the methods of producing a decellularized tissue scaffold is performed at about between 1 hours to 48 hours.

In one embodiment, step (ii) of the methods of producing a decellularized tissue scaffold disclosed herein is performed at 4° C. for 24 hours. In another embodiment, step (ii) of the methods of producing a decellularized tissue scaffold is performed at about 4° C. for about 24 hours. In another embodiment, step (ii) of the methods of producing a decellularized tissue scaffold is performed at about between 0° C. and 10° C. In another embodiment, step (ii) of the methods of producing a decellularized tissue scaffold is performed at about between 1 hour and 48 hours.

In one embodiment, step (iii) of the methods of producing a decellularized tissue scaffold disclosed herein is performed at 37° C. for 48 hours. In another embodiment, step (iii) of the methods of producing a decellularized tissue scaffold is performed at about 37° C. for about 48 hours. In another embodiment, step (iii) of the methods of producing a decellularized tissue scaffold is performed at about between 20° C. to 51° C. In another embodiment, step (iii) of the methods of producing a decellularized tissue scaffold is performed at about between 24 and 72 hours.

In one embodiment, step (iv) of the methods of producing a decellularized tissue scaffold disclosed herein is performed at 37° C. for 30 minutes. In another embodiment, step (iv) of the methods of producing a decellularized tissue scaffold is performed at about 37° C. for about 30 minutes. In another embodiment, step (iv) of the methods of producing a decellularized tissue scaffold is performed at about between 20° C. to 51° C. In another embodiment, step (iv) of the methods of producing a decellularized tissue scaffold is performed at about between 5 minutes to 2 hours.

In one embodiment, step (v) of the methods of producing a decellularized tissue scaffold disclosed herein is performed at 4° C. for 38 hours. In another embodiment, step (v) of the methods of producing a decellularized tissue scaffold is performed at about 4° C. for about 38 hours. In another embodiment, step (v) of the methods of producing a decellularized tissue scaffold is performed at about between 0° C. and 10° C. In another embodiment, step (v) of the methods of producing a decellularized tissue scaffold is performed at about between 20 hours to 76 hours.

In one embodiment, step (vi) of the methods of producing a decellularized tissue scaffold disclosed herein is performed at 37° C. for 48 hours. In another embodiment, step (vi) of the methods of producing a decellularized tissue scaffold disclosed herein is performed at about 37° C. for about 48 hours. In another embodiment, step (vi) of the methods of producing a decellularized tissue scaffold is performed at about between 20° C. to 51° C. In another embodiment, step (vi) of the methods of producing a decellularized tissue scaffold is performed at about between 24 and 72 hours.

In one embodiment, step (vii) of the methods of producing a decellularized tissue scaffold disclosed herein is performed at 4° C. for 48 hours. In another embodiment, step (vii) of the methods of producing a decellularized tissue scaffold disclosed herein is performed at about 4° C. for about 48 hours. In another embodiment, step (vii) of the methods of producing a decellularized tissue scaffold disclosed herein is performed at about between 0° C. and 10° C. In another embodiment, step (vii) of the methods of producing a decellularized tissue scaffold disclosed herein is performed at about between 24 and 72 hours.

In one embodiment, step (vii) of the methods of producing a decellularized tissue scaffold disclosed herein is performed to remove the remaining detergent agents. In one embodiment, step (vii) of the methods of producing a decellularized tissue scaffold disclosed herein is performed to remove the detergent agents.

In one embodiment, step (viii) of the methods of producing a decellularized tissue scaffold disclosed herein is performed at 4° C. In another embodiment, step (viii) of the methods of producing a decellularized tissue scaffold disclosed herein is performed at about 4° C. In another embodiment, step (viii) of the methods of producing a decellularized tissue scaffold disclosed herein is performed at between about 0° C. to 10° C.

In one embodiment, disclosed herein is a method of producing a decellularized tissue scaffold comprising (i) rinsing a tissue sample with cation detergent, for example 1% Triton X-100; (ii) washing said sample with phosphate buffered saline (PBS); (iii) preserving said sample in 1% sodium dodecyl sulfate (SDS); (iv) preserving said sample in 0.05% Trypsin solution; (v) rinsing said sample with PBS; (vi) using 2% SDS solution on said sample; and (vii) washing said sample with PBS, thereby producing a decellularized tissue scaffold.

In one embodiment, disclosed herein is a method of producing a decellularized prostate tissue scaffold comprising (i) rinsing a prostate tissue sample with cation detergent, for example 1% Triton X-100: (ii) washing said sample with phosphate buffered saline (PBS); (iii) preserving said sample in 1% sodium dodecyl sulfate (SDS); (iv) preserving said sample in 0.05% Trypsin solution; (v) rinsing said sample with PBS; (vi) using 2% SDS solution on said sample; and (vii) washing said sample with PBS, thereby producing a decellularized tissue scaffold.

In one embodiment, disclosed herein is a method of producing a decellularized human prostate tissue scaffold comprising (i) rinsing a human prostate tissue sample with cation detergent, for example 1% Triton X-100; (ii) washing said sample with phosphate buffered saline (PBS); (iii) preserving said sample in 1% sodium dodecyl sulfate (SDS); (iv) preserving said sample in 0.05% Trypsin solution; (v) rinsing said sample with PBS; (vi) using 2% SDS solution on said sample; and (vii) washing said sample with PBS, thereby producing a decellularized tissue scaffold.

In one embodiment, disclosed herein is a method of producing a decellularized human prostate tissue scaffold comprising: (i) rinsing a tissue sample with 1% Triton X-100 at 37° C. for 24 hours; (ii) washing said sample with phosphate buffered saline (PBS) at 4° C. for 24 hours; (iii) preserving said sample in 1% sodium dodecyl sulfate (SDS) at 37° C. for 48 hours; (iv) preserving said sample in 0.05% Trypsin solution on a shaker at 37° C. for 30 minutes; (v) rinsing said sample with PBS at 4° C. for 48 hours; (vi) using 2% SDS solution on said sample at 37° C. for 48 hours; and (vii) washing said sample with PBS at 4° C. for 48 hours; thereby producing a decellularized tissue scaffold.

In one embodiment, disclosed herein is a method of producing a decellularized human prostate tissue scaffold comprising: (i) rinsing a tissue sample with 1% Triton X-100 at 37° C. for 24 hours; (ii) washing said sample with phosphate buffered saline (PBS) at 4° C. for 24 hours; (iii) preserving said sample in 1% sodium dodecyl sulfate (SDS) at 37° C. for 48 hours; (iv) preserving said sample in 0.05% Trypsin solution on a shaker at 37° C. for 30 minutes; (v) rinsing said sample with PBS at 4° C. for 48 hours; (vi) using 2% SDS solution on said sample at 37° C. for 48 hours; (vii) washing said sample with PBS at 4° C. for 48 hours; thereby producing a decellularized tissue scaffold; and (viii) preserving the decellularized prostate tissue scaffold in an antibiotic solution at 4° C. In another embodiment, the antibiotic solution comprises penicillin, gentamycin, and ceftriaxone.

In one embodiment, disclosed herein is a method of producing a decellularized human tissue scaffold comprising: (i) rinsing a tissue sample with 1% Triton X-100 at 37° C. for 24 hours; (ii) washing said sample with phosphate buffered saline (PBS) at 4° C. for 24 hours; (iii) preserving said sample in 1% sodium dodecyl sulfate (SDS) at 37° C. for 48 hours; (iv) preserving said sample in 0.05% Trypsin solution on a shaker at 37° C. for 30 minutes; (v) rinsing said sample with PBS at 4° C. for 48 hours; (vi) using 2% SDS solution on said sample at 37° C. for 48 hours; and (vii) washing said sample with PBS at 4° C. for 48 hours; thereby producing a decellularized tissue scaffold.

In one embodiment, disclosed herein is a method of producing a decellularized human tissue scaffold comprising: (i) rinsing a tissue sample with 1% Triton X-100 at 37° C. for 24 hours; (ii) washing said sample with phosphate buffered saline (PBS) at 4° C. for 24 hours; (iii) preserving said sample in 1% sodium dodecyl sulfate (SDS) at 37° C. for 48 hours; (iv) preserving said sample in 0.05% Trypsin solution on a shaker at 37° C. for 30 minutes; (v) rinsing said sample with PBS at 4° C. for 48 hours; (vi) using 2% SDS solution on said sample at 37° C. for 48 hours; (vii) washing said sample with PBS at 4° C. for 48 hours; thereby producing a decellularized tissue scaffold; and (viii) preserving the decellularized tissue scaffold in an antibiotic solution at 4° C. In another embodiment, the antibiotic solution comprises penicillin, gentamycin, and ceftriaxone.

In one embodiment, the decellularized human prostate tissue scaffold produced by the methods disclosed herein are divided into several segments.

In one embodiment, the decellularized human prostate tissue scaffold produced by the methods disclosed herein are preserved at 4° C. In another embodiment, the decellularized human prostate tissue scaffold produced by the methods disclosed herein are preserved at about 4° C. In another embodiment, the decellularized human prostate tissue scaffold produced by the methods disclosed herein are preserved at between about 0° C. to 10° C.

In one embodiment, the decellularized human prostate tissue scaffold produced by the methods disclosed herein are stored at 4° C. In another embodiment, the decellularized human prostate tissue scaffold produced by the methods disclosed herein are stored at about 4° C. In another embodiment, the decellularized human prostate tissue scaffold produced by the methods disclosed herein are stored at between about 0° C. to 10° C.

In one embodiment, the continuous shaking is performed with a mechanical shaker. In another embodiment, the continuous shaking is performed by hand. In another embodiment, the continuous shaking is done by mechanical means. In another embodiment, the continuous shaking is done by a continuous stirred tank reactor. In another embodiment, the continuous shaking is done by a rotary shaker.

In one embodiment, the continuous shaking performed by a mechanical shaker is regulated to circulate at 70 rpm. In another embodiment, the continuous shaking performed by the mechanical shaker is regulated to circulate at any value between 60 rpm and 80 rpm. In another embodiment, the continuous shaking performed by the mechanical shaker is regulated to circulate at any value between 50 rpm and 90 rpm. In another embodiment, the continuous shaking performed by the mechanical shaker is regulated to circulate at any value between 50 rpm and 90 rpm. In another embodiment, the continuous shaking performed by the mechanical shaker is regulated to circulate at any value between 40 rpm and 100 rpm. In another embodiment, the continuous shaking performed by the mechanical shaker is regulated to circulate at any value between 100 rpm and 200 rpm.

In one embodiment, the mechanical shaker has a speed of 70 rpm. In another embodiment, the mechanical shaker has a speed of any value between 60 rpm and 80 rpm. In another embodiment, the mechanical shaker has a speed of any value between 50 rpm and 90 rpm. In another embodiment, the mechanical shaker has a speed of any value between 50 rpm and 90 rpm. In another embodiment, the mechanical shaker has a speed of any value between 40 rpm and 100 rpm. In another embodiment, the mechanical shaker has a speed of any value between 100 rpm and 200 rpm.

In one embodiment, the tissue sample provided to produce a decellularized human tissue scaffold is from a cadaver. In another embodiment, the tissue sample provided to produce a decellularized human tissue scaffold is from a human cadaver. In another embodiment, the tissue sample provided to produce a decellularized human tissue scaffold is from a human subject. In another embodiment, the tissue sample provided to produce a decellularized human tissue scaffold is harvested from a human subject. In another embodiment, the tissue sample provided to produce a decellularized human tissue scaffold is harvested from a living human subject. In another embodiment, the tissue sample provided to produce a decellularized human tissue scaffold is from a human male cadaver or living human male subject. In another embodiment, the tissue sample provided to produce a decellularized human tissue scaffold is harvested from a living human male subject. In another embodiment, the tissue sample provided to produce a decellularized human tissue scaffold is harvested from a human source.

In one embodiment, the tissue sample is biologically natural. In another embodiment, the tissue sample is normal. In another embodiment, the tissue sample is malignant. In another embodiment, the tissue sample is benign.

In one embodiment, the organ sample provided to produce a decellularized human organ scaffold is from a cadaver. In another embodiment, the organ sample provided to produce a decellularized human organ scaffold is from a human cadaver. In another embodiment, the organ sample provided to produce a decellularized human organ scaffold is from a human subject. In another embodiment, the organ sample provided to produce a decellularized human organ scaffold is harvested from a human subject. In another embodiment, the organ sample provided to produce a decellularized human organ scaffold is harvested from a living human subject. In another embodiment, the organ sample provided to produce a decellularized human organ scaffold is from a human male cadaver or living human male subject. In another embodiment, the organ sample provided to produce a decellularized human organ scaffold is harvested from a living human male subject. In another embodiment, the organ sample provided to produce a decellularized human organ scaffold is harvested from a human source.

In one embodiment, the organ sample is biologically natural. In another embodiment, the organ sample is normal. In another embodiment, the organ sample is malignant. In another embodiment, the organ sample is benign.

In one embodiment, the prostate tissue sample provided to produce a decellularized human prostate tissue scaffold is from a cadaver. In another embodiment, the prostate tissue sample provided to produce a decellularized human prostate tissue scaffold is from a human cadaver. In another embodiment, the prostate tissue sample provided to produce a decellularized human prostate tissue scaffold is from a human subject. In another embodiment, the prostate tissue sample provided to produce a decellularized human prostate tissue scaffold is harvested from a human subject. In another embodiment, the prostate tissue sample provided to produce a decellularized human prostate tissue scaffold is harvested from a living human subject. In another embodiment, the prostate tissue sample provided to produce a decellularized human prostate tissue scaffold is from a human male cadaver or living human male subject. In another embodiment, the prostate tissue sample provided to produce a decellularized human prostate tissue scaffold is harvested from a living human male subject. In another embodiment, the prostate tissue sample provided to produce a decellularized human prostate tissue scaffold is harvested from a human source.

In one embodiment, the prostate tissue or organ sample is biologically natural. In another embodiment, the prostate tissue or organ sample is normal. In another embodiment, the prostate tissue or organ sample is malignant. In another embodiment, the prostate tissue or organ sample is benign. In another embodiment, the prostate tissue or organ sample is from an enlarged prostate gland. In another embodiment, the prostate tissue or organ sample is from an enlarged prostate gland due to benign prostatic hyperplasia (BPH). In another embodiment, the prostate tissue or organ sample is a Gleason grade 2, 3, 4, or 5 cancer sample.

In one embodiment, the tissue sample is provided in a phosphate buffered saline (PBS). In another embodiment, the tissue sample is washed in a phosphate buffered saline (PBS) prior to decellularizing. In another embodiment, the tissue sample is washed in a phosphate buffered saline (PBS) for about 48 hours prior to decellularizing. In another embodiment, the tissue sample is washed in a phosphate buffered saline (PBS) for about 24 hours prior to decellularizing. In another embodiment, the tissue sample is washed in a phosphate buffered saline (PBS) for anywhere between 24 and 72 hours prior to decellularizing.

In one embodiment, the prostate tissue sample is provided in a phosphate buffered saline (PBS). In another embodiment, the prostate tissue sample is washed in a phosphate buffered saline (PBS) prior to decellularizing. In another embodiment, the prostate tissue sample is washed in a phosphate buffered saline (PBS) for about 48 hours prior to decellularizing. In another embodiment, the prostate tissue sample is washed in a phosphate buffered saline (PBS) for about 24 hours prior to decellularizing. In another embodiment, the prostate tissue sample is washed in a phosphate buffered saline (PBS) for anywhere between 24 and 72 hours prior to decellularizing.

In one embodiment, the tissue sample or prostate tissue sample is sliced prior to decellularizing. In one embodiment, the tissue sample or prostate tissue sample is sliced prior to washing in PBS. In one embodiment, the tissue sample or prostate tissue sample is sliced after washing in PBS. In another embodiment, the prostate tissue sample is provided in a phosphate buffered saline (PBS), is sliced, and is washed with PBS for 48 hours

In one embodiment, the compositions used in the methods described herein can be formulated in water or phosphate buffered saline, e.g., as a solution or suspension. In another embodiment, the compositions are formulated in phosphate buffered saline.

In one embodiment, the detergent used to produce a decellularized tissue or organ scaffold is a cationic detergent. In some embodiments, the detergent is a cationic detergent such as a quaternary ammonium detergent. In some embodiments, the detergent is a non-ionic detergent such as ethoxylates such as Tween and Triton. In some embodiments, the detergent is sodium dodecyl sulfate (SDS). Other detergents or other chemicals are also possible and those skilled in the art would be capable of selecting suitable detergents based upon the teachings of the specification.

In one embodiment, the detergent used to produce a decellularized tissue or organ scaffold is a cationic detergent selected from the group consisting of Triton X-100, Triton X-200, Tween 20, Tween 80, sodium deoxycholate. CHAPS, sodium dodecyl sulfate (SDS), N-lauroyl-sarcosinate, Igepal CA630, and Sulfobetain-10 and -16, or any combination thereof.

In one embodiment, the detergent used to produce decellularized tissue or organ scaffold is selected from the group consisting of 1% Triton X-100, 1% sodium dodecyl sulfate (SDS), and 0.05% Trypsin solution, or any combination thereof. In one embodiment, the detergent used to produce decellularized tissue or organ scaffold is selected from the group consisting of 1% (v/v) Triton X-100, 1% (v//v) sodium dodecyl sulfate (SDS), and 0.05% (v/v) Trypsin solution, or any combination thereof. In another embodiment, the detergent used to produce decellularized tissue or organ scaffold is 1% (v/v) Triton X-100.

In one embodiment all percentages disclosed herein are v/v.

In one embodiment, enzymes can be used to accomplish decellularization, including but not limited to dispase II, trypsin, and thermolysin. Trypsin attacks the desmosome complex between cells.

In one embodiment, following the treating of the human tissue sample with a detergent to produce a decellularized human tissue scaffold, the decellularized human tissue scaffold is prepared in a manner to prevent microbial contamination. In another embodiment, the prevention of microbial contamination in the decellularized human tissue scaffold is accomplished by preparing all solutions in distilled water and/or autoclaving. In another embodiment, the prevention of microbial contamination in the decellularized human tissue scaffold is accomplished by autoclaving. In another embodiment, the prevention of microbial contamination in the decellularized human tissue scaffold is accomplished by preparing all solutions in 1% antibiotic/mycotic. In another embodiment, the decellularized human tissue scaffold is washed with sterile distilled water to remove any residual cellular debris and chemicals.

In one embodiment, following the treating of the human tissue sample with a detergent to produce a decellularized human tissue scaffold, the decellularized human tissue scaffold is preserved in an antibiotic solution. In another embodiment, the antibiotic solution contains 1% antibiotic/mycotic. In another embodiment, the antibiotic solution contains distilled water. In another embodiment, the antibiotic solution comprises penicillin, gentamycin, and ceftriaxone, or any combination thereof. In another embodiment, the antibiotic solution is a pre-mixed antibiotic solution. In another embodiment, the premixed antibiotic solution is a cocktail of antibiotics. In another embodiment, the premixed antibiotic solution is a cocktail of antibiotics such as Streptomycin Sulfate or Gentamicin Sulfate. In another embodiment, other antibiotics such as Polymixin B Sulfate, Bacitracin, or similar antibiotics now available or available in the future, are also suitable.

In one embodiment, preserving the decellularized human tissue scaffold prevents microbial contamination of the decellularized organ scaffold. In another embodiment, preserving the decellularized human tissue scaffold by treatment with an antibiotic solution or cocktail prevents microbial contamination of the decellularized organ scaffold.

In one embodiment, the decellularized human tissue scaffold is sterilized. In another embodiment, the decellularized human tissue scaffold is sterilized to removes residual cellular debris and chemicals. In another embodiment, cellular debris and chemicals are removed from the decellularized human tissue scaffold. In another embodiment, the decellularized human tissue scaffold is sterilized by washing the decellularized organ tissue scaffolds with sterile distilled water.

In one embodiment, following the treating of the human prostate tissue sample with a detergent to produce a decellularized human prostate tissue scaffold, the decellularized human prostate tissue scaffold is prepared in a manner to prevent microbial contamination. In another embodiment, the prevention of microbial contamination in the decellularized human prostate tissue scaffold is accomplished by preparing all solutions in distilled water and/or autoclaving. In another embodiment, the prevention of microbial contamination in the decellularized human prostate tissue scaffold is accomplished by autoclaving. In another embodiment, the prevention of microbial contamination in the decellularized human prostate tissue scaffold is accomplished by preparing all solutions in 1% antibiotic/mycotic. In another embodiment, the decellularized human prostate tissue scaffold is washed with sterile distilled water to remove any residual cellular debris and chemicals.

In one embodiment, following the treating of the human prostate tissue sample with a detergent to produce a decellularized human prostate tissue scaffold, the decellularized human prostate tissue scaffold is preserved in an antibiotic solution. In another embodiment, the antibiotic solution contains 1% antibiotic/mycotic. In another embodiment, the antibiotic solution contains distilled water. In another embodiment, the antibiotic solution comprises penicillin, gentamycin, and ceftriaxone, or any combination thereof. In another embodiment, the antibiotic solution is a pre-mixed antibiotic solution. In another embodiment, the premixed antibiotic solution is a cocktail of antibiotics. In another embodiment, the premixed antibiotic solution is a cocktail of antibiotics such as Streptomycin Sulfate or Gentamicin Sulfate. In another embodiment, other antibiotics such as Polymixin B Sulfate, Bacitracin, or similar antibiotics now available or available in the future, are also suitable.

In one embodiment, preserving the decellularized human prostate tissue scaffold prevents microbial contamination of the decellularized organ scaffold. In another embodiment, preserving the decellularized human prostate tissue scaffold by treatment with an antibiotic solution or cocktail prevents microbial contamination of the decellularized organ scaffold.

In one embodiment, the decellularized human prostate tissue scaffold is sterilized. In another embodiment, the decellularized human prostate tissue scaffold is sterilized to removes residual cellular debris and chemicals. In another embodiment, cellular debris and chemicals are removed from the decellularized human prostate tissue scaffold. In another embodiment, the decellularized human prostate tissue scaffold is sterilized by washing the decellularized organ prostate tissue scaffolds with sterile distilled water.

In one embodiment, the malignant tissue samples reveal prominent decreases in type IV Collagen content of the decellularized tissue scaffold. In one embodiment, the malignant prostate tissue samples reveal prominent decreases in type IV Collagen content of the decellularized prostate tissue scaffold.

In one embodiment, the decellularized tissue scaffold has a decrease in laminin and vimentin compared to the tissue sample. In one embodiment, the decellularized prostate tissue scaffold has a decrease in laminin and vimentin compared to the prostate tissue sample.

In one embodiment, the methods described here further comprise repopulating the decellularized tissue scaffold with cells to produce a recellularized tissue scaffold. In one embodiment, the methods described here further comprise repopulating the decellularized organ scaffold with cells to produce a recellularized organ scaffold. In one embodiment, the methods described here further comprise repopulating the decellularized prostate tissue scaffold with cells to produce a recellularized prostate tissue scaffold.

In one embodiment, the methods described here further comprise recellularizing the decellularized tissue scaffold in-vitro. In one embodiment, the methods described here further comprise recellularizing the decellularized organ scaffold in-vitro. In one embodiment, the methods described here further comprise recellularizing the decellularized prostate tissue scaffold in-vitro.

In one embodiment, the methods described here further comprise recellularizing the decellularized tissue scaffold in-vivo. In one embodiment, the methods described here further comprise recellularizing the decellularized organ scaffold in-vivo. In one embodiment, the methods described here further comprise recellularizing the decellularized prostate tissue scaffold in-vivo.

In one embodiment, the methods described here further comprise recellularizing the decellularized tissue scaffold by in-vivo implantation of the decellularized tissue scaffold in a human subject. In another embodiment, the methods described here further comprise recellularizing the decellularized organ scaffold by in-vivo implantation of the decellularized organ scaffold in a human subject. In another embodiment, the methods described here further comprise recellularizing the decellularized prostate tissue scaffold by in-vivo implantation of the decellularized prostate tissue scaffold in a human subject.

In one embodiment, the in-vivo implantation induces cell proliferation in the decellularized tissue scaffold. In one embodiment, the in-vivo implantation induces cell proliferation in the decellularized organ scaffold. In one embodiment, the in-vivo implantation induces cell proliferation in the decellularized prostate tissue scaffold.

In one embodiment, the in-vivo implantation induces cell vascularization in the decellularized tissue scaffold. In one embodiment, the in-vivo implantation induces cell vascularization in the decellularized organ scaffold. In one embodiment, the in-vivo implantation induces cell vascularization in the decellularized prostate tissue scaffold.

In one embodiment, following recellularization of the tissue scaffold, the recellularized tissue does not contain cancerous cells. In another embodiment, following recellularization of the organ scaffold, the recellularized organ does not contain cancerous cells.

In another embodiment, following recellularization of the prostate tissue scaffold, the recellularized prostate tissue does not contain cancerous cells.

In one embodiment, the absence of cancerous cells in the recellularized tissues is confirmed by immunostaining of the prostate tissue scaffolds that are negative for AMACR marker.

In one embodiment, a decellularized tissue scaffold is produced by any of the methods disclosed herein. In another embodiment, a decellularized organ scaffold is produced by any of the methods disclosed herein. In one embodiment, a decellularized prostate tissue scaffold is produced by any of the methods disclosed herein.

In one embodiment, an extracellular matrix is produced by any of the methods disclosed herein. In another embodiment, an extracellular matrix is produced by any of the methods disclosed herein. In one embodiment, an extracellular matrix is produced by any of the methods disclosed herein.

In one embodiment, a decellularized tissue scaffold is produced by any of the methods disclosed herein. In another embodiment, a decellularized organ scaffold is produced by any of the methods disclosed herein. In one embodiment, a decellularized prostate tissue scaffold is produced by any of the methods disclosed herein.

In one embodiment, a recellularized tissue scaffold is produced by any of the methods disclosed herein. In another embodiment, a recellularized organ scaffold is produced by any of the methods disclosed herein. In one embodiment, a recellulanzed prostate tissue scaffold is produced by any of the methods disclosed herein.

In one embodiment, disclosed herein is a method of treating a disorder of the prostate by implanting the decellularized prostate tissue scaffolds produced by the methods of disclosed in a human subject. In another embodiment, the disorder of the prostate is benign prostatic hyperplasia (BPH). In another embodiment, the disorder of the prostate is benign prostate cancer.

Summarizing the above, standard techniques such as the above mentioned surgery, radiation therapy, hormonal therapy, occasionally chemotherapy, proton therapy, etc., if applied alone, do not appear to be suitable for an efficient treatment of prostate cancer (PCa).

Some aspects of this disclosure provide methods and reagents for the preparation of decellularized prostate tissue scaffold from tissues or organs harvested from a subject or cadavers. Some aspects of this disclosure provide methods and reagents for removing cancer cells within such decellularized biomatrices. Some aspects of this invention provide methods and reagents for the generation of tissue constructs comprising a decellularized prostate tissue scaffold. Some aspects of this invention provide bioreactors for the generation of tissue constructs. Some aspects of this invention provide methods and reagents for the analysis and evaluation of cancer-associated characteristics in tissue constructs.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

### Example 1

### Decellularization and Recellularization of Normal, BPH, and Malignant Prostate Tissues

Objective: Development of an efficient protocol for decellularization of natural, benign prostatic hyperplasia (BPH), and malignant human prostatic tissues. Furthermore, the components and structural characteristics of the provided scaffolds were investigated to illustrate the ECM alterations and variations between the scaffolds. Subsequently, the influence of collagenous ECM on cell behavior and alteration in expression of ECM components were studied by implanting the scaffolds in male rats, adjacent to the animals' prostate. The cell seeding process of the decellularized tissues as well as the properties of the different recellularized samples were examined.

In this study, all the animals' procedures were performed in compliance with the regulations of Guide for the Care and Use of Laboratory Animals prepared by the Institute of Laboratory Animal Resources, National Research Council (NIH Publications No. 8023, revised 1978). The Animal Ethics Committee of the Tehran University of Medical Sciences, School of Medicine and Education Section of Basic Sciences, approved the animal selection, managements, and surgical protocols.

### Scaffold Preparation

After obtaining informed consent, natural, benign prostatic hyperplasia and malignant human prostatic tissues were obtained. Normal prostatic tissues were aseptically prepared from six brain-dead patients. Six BPH and malignant tissues were also obtained from patients undergoing transurethral prostate resection/radical prostatectomy. The specimens were evaluated for any histological changes or Gleason scores of the malignant samples.

The harvested tissues were aseptically transferred to the laboratory in phosphate buffered saline (PBS); were sliced and washed with PBS for 48 hours

The decellularization process was initiated by rinsing the specimens with 1% Triton X-100 on a shaker plate at 37° C. for 24 hours. The samples were washed with PBS at 4° C. for 24 hours. Subsequently, they were preserved in 1% sodium dodecyl sulfate (SDS) at 37° C. for 48 hours.

In the next step, the specimens were preserved in 0.05% Trypsin solution for 30 minutes on the shaker at 37° C., followed by another 48 hours of PBS rinsing at 4° C. Finally, we used 2% SDS solution at 37° C. for 48 hours, and the samples were washed with PBS at 4° C. for 48 hours to remove the remaining detergent agents.

All of the previous steps were performed under continuous shaking with the mechanical shaker regulated to circulate at 70 rpm.

The decellularized prostates were divided into several segments and preserved in an antibiotic solution containing penicillin, gentamycin, and ceftriaxone at 4° C. for further implantation.

### DAPI Staining and DNA Quantification

4, 6-diamidino-2-phenylindole (DAPI) staining was performed to evaluate double-stranded DNA content of both natural and decellularized specimens and determine the efficacy of the decellularization protocol in nuclear components removal. In order to perform DAPI staining, sections were deparaffinized, and were incubated in 1 μg/ml DAPI solution (Sigma, St Louis, Mo., USA) for 15 minutes. Subsequently, the samples were washed with PBS for 15 minutes and were observed by a fluorescence microscope using a UV filter. Slices were assessed with an inverted fluorescence microscope (Diaphot 200; Nikon, Tokyo, Japan) after rinsing in dH2O. The images were taken by a specialist who was blind to the study. Moreover. DNA quantification assay was carried out using a genomic DNA purification kit (Thermo Scientific, Lithuania) and Nanodrop spectrophotometry (Thermo scientific Nanodrop 1000) to measure the DNA concentration of the samples. Total double-stranded DNA content (ng/ml lysate) of native and decellularized tissues were extracted, quantified, and normalized to the dry weight of each specimen.

### Scanning Electron Microscopy (SEM)

SEM was performed in order to examine the structural features of the scaffolds before implantation and evaluate the scaffold surface morphology and thickness. The samples were fixed by 2.5% glutaraldehyde (Merck, Darmstadt, Germany) and were rinsed with PBS solution three times for a total of 45 minutes at 3° C. Subsequently, they were dehydrated in graded series of Ethanol (30%, 50%, 70%, 90% and 100%), subjected to critical point drying, and gold sputter coating layer (approximately 3 nm thick). Finally, the images were provided via SEM (S3500N; Hitachi High Technologies America) to demonstrate the impact of decellularization process on the ultra-structure of the tissues and to compare the ECM arrangement in natural, BPH and cancerous samples.

### TUNEL Assay

TUNEL (terminal deoxynucleotidyl transferase dUTP nick end labeling) assay was performed to detect the apoptotic cells before and after the decellularization process. In-situ cell death detection kit, Fluorescein (Roche applied science, Penzberg, Germany) was used for detection of apoptosis at single cell level, and the data was quantified using Image J software.

### Surgical Technique

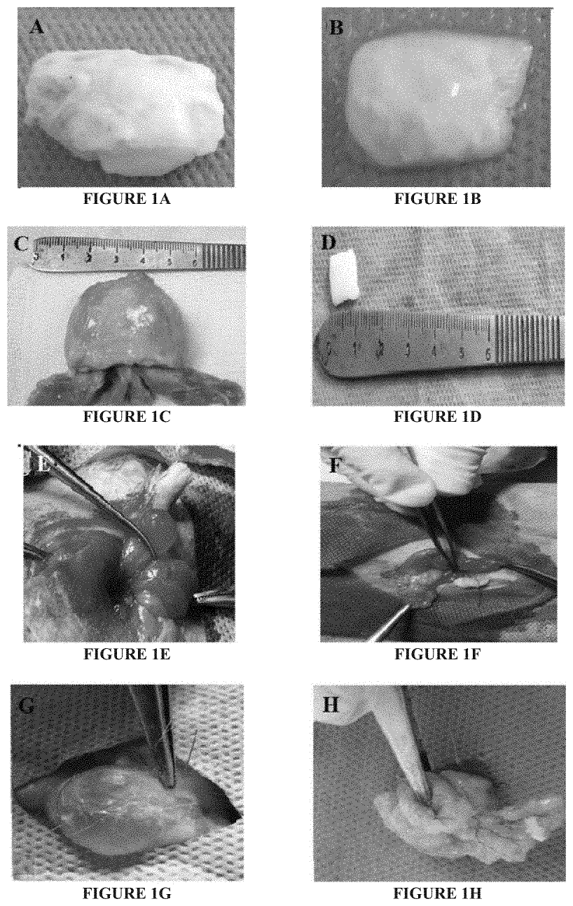

Nine male GFP-positive Wistar rats (200-250 g) were anesthetized using 5 mg/kg Xylazine and 0.3 mg/kg Ketamine and positioned in lithotomy position and then shaved to expose the surgical region. We made a lower abdominal incision were made, and the skin and the fascia were dissected to expose the bladder. The anatomical location of the prostate was detected which was considered as the implant site. The animals were randomly divided into three groups. Natural, BPH, and malignant prostate scaffolds were implanted in rats of group A (n=3), group B (n=3), and group C (n=3), respectively. For this purpose, 1 cm³ sections of the scaffolds were placed adjacent to prostate and surrounded the decellularized tissues with the testicular omentum to establish a sandwich-like structure which previously demonstrated a considerable recellularizing potential as a natural bio-reactor. Finally, the grafts were fixed by 6.0 prolene sutures, and 4.0 prolene stitches were used for the muscular layer (FIGS. 1A-1H).

Daily Cefazolin was used intramuscularly for all animals for four consecutive days after the surgery. The three groups were assessed independently for 6 months after implantation to assess the quality of recellularization by histological evaluations.

### Histological and Immunohistochemical (IHC) Examinations

In order to evaluate the efficacy of the decellularization process and estimate the results of in vivo regeneration, decellularized and implanted prostate scaffolds were fixed in 10% natural buffered formalin. Samples were paraffin embedded, cut into 5 μm sections, and stained by hematoxylin and eosin (H&E) and Masson's Trichrome. In addition, sections were immersed in Triton X-100 in 1:100 for 15 minutes at room temperature. IHC studies were carried out using markers including Fibronectin, Laminin, Vimentin, and Collagen types I, III, and IV specific antibodies to evaluate the expression of ECM components before and after the decellularization process. Moreover, we performed IHC staining for P63, Alpha-methylacyl-CoA racemase (AMACR), CKAE1/AE3+, PSA−, CK7−, CK20−, LCA, CD68−, and CD34+ in the groups after the implantation, to determine the efficacy of the recellularization process, illustrate the cell types, and to compare the cell population in different groups. High expression of AMACR marker and decreased level of P63-positive cells were considered as signs of malignant cells presence.

A Nikon digital camera DXM 1200 (Amsterdam. Netherlands) was used to obtain the images. Photoshop 10.0 software (Adobe Systems, Inc., Mountain View, Calif., USA) and Image Pro (Image Pro Inc., Boston, Mass., USA) were used for image analysis. For scoring the images, five photomicrographs (X100) were used and the mean scores were used as final values for analysis.

### Statistical Analysis

Statistical Package for the Social Sciences (SPSS; version 20, SPSS Inc., Chicago, Ill., USA) was used for statistical analysis of the data. Numerical results were expressed as mean±standard deviation and were tested by one-way ANOVA. The level of significance was considered as a P value of less than 0.05.

### Results

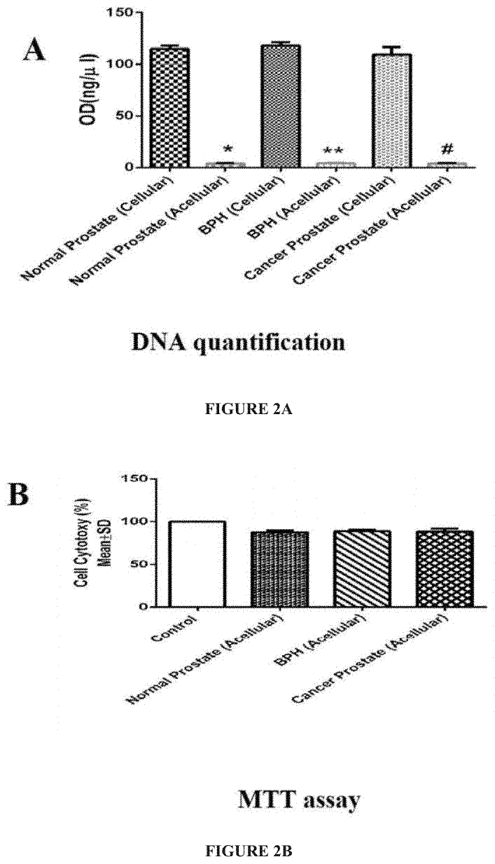

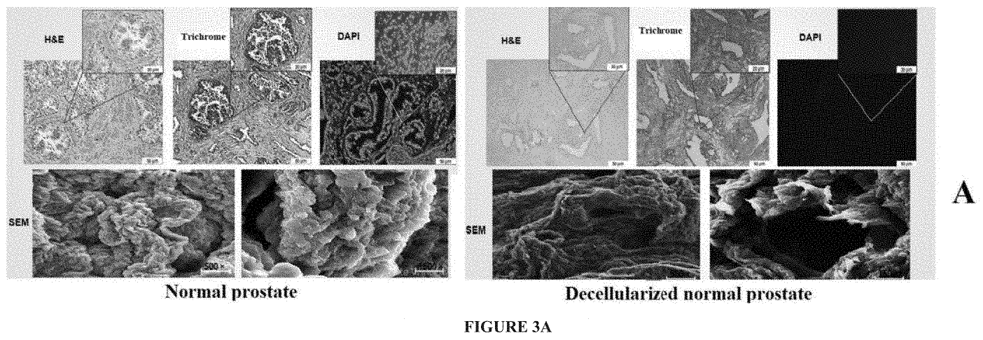

H&E staining of normal BPH tissues revealed prominent stromal proliferation with increased smooth muscle fibers as well as enlarged prostatic glands, which was also maintained in post-decellularization examinations. The glandular structures were maintained in peripheral and central regions in both specimens. The malignant prostate samples were obtained from patients with a previous diagnosis of cancer and dominating patterns of grade 3 and 4 in Gleason grading system. Enlarged cancerous glands with alternating morphologies that were distinguishable by cellular atypia and lack of basal cells and corpora amylacea were also observed. In contrast to natural and BPH samples, cancerous samples revealed a high concentration of AMACR-positive cells confirming the presence of malignant cells before decellularization process. Furthermore, immunostaining of P63 that was highly expressed in basal cells of non-malignant prostatic tissue resulted in negative outcomes in the malignant samples before the decellulanzation. The results of all decellularized samples were indicative of complete removal of malignant and non-malignant cells. Nuclear components were homogenously removed from all specimens with no variations between peripheral and central areas. Quantification tests also showed less than 5 ng/ul of DNA in all three groups after the decellularization process indicative of complete cell removal in all specimens with no significant difference between three groups (P-value>0.05) (FIG. 2A). MTT assay was performed to determine the toxicity and bio-compatibility of the provided bio-scaffolds. All the samples showed a viability of 90% or higher following the decellularization process. Additionally, no significant differences were detected between Normal, BPH, malignant scaffolds and the control samples (p<0.05) (FIG. 2B).

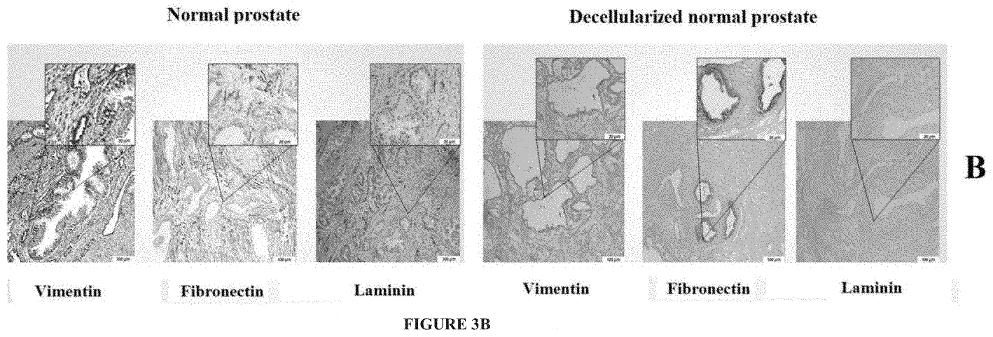

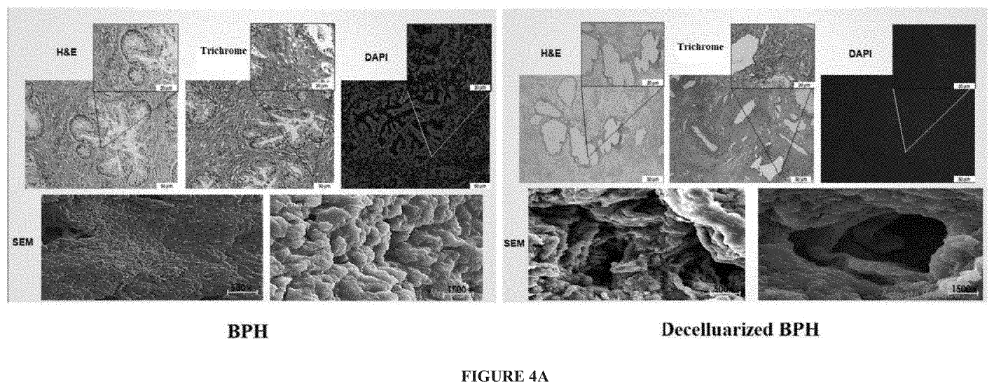

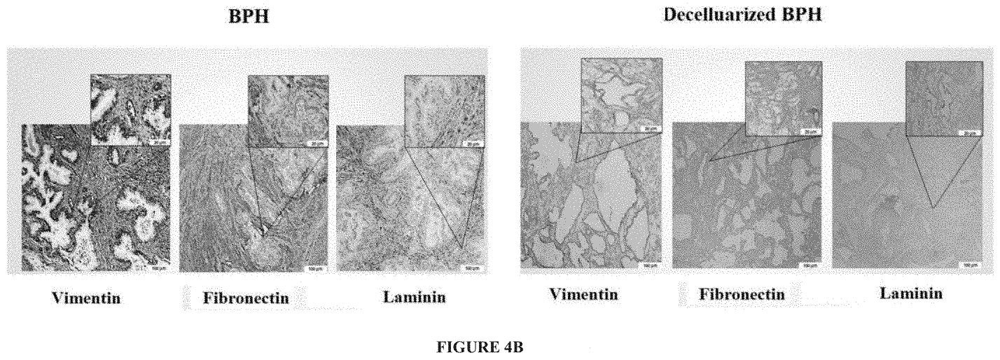

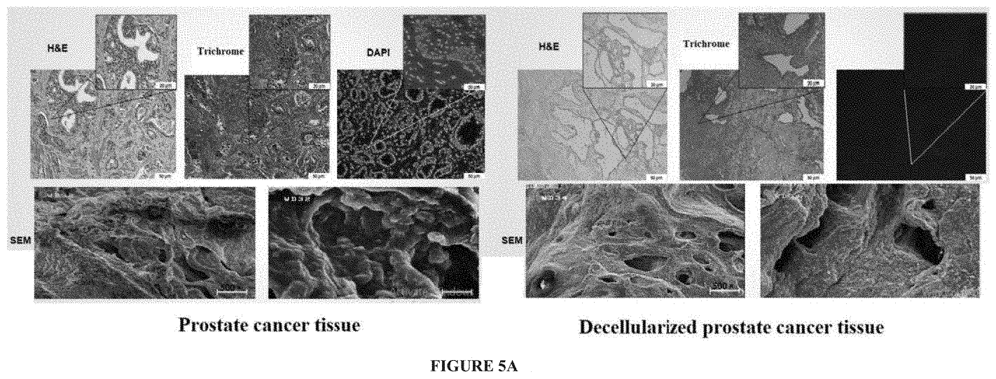

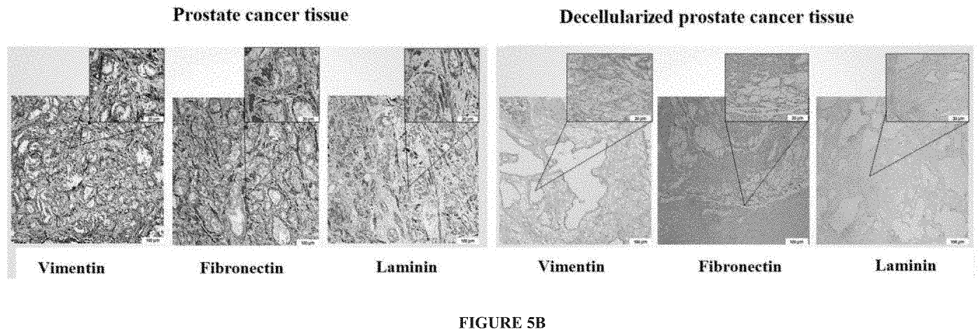

IHC staining revealed that the expression of laminin and vimentin was down-regulated after decellularization process in normal, BPH, and malignant tissues. The higher fibronectin content before decellularization process was observed in BPH tissue (88.75±0.25 vs. 61.25±0.75 and 45.25±0.25 in normal and cancerous tissue, respectively). However, fibronectin expression was decreased in all the samples after decellulanzation with the lowest expression in malignant tissue followed by normal and BPH specimens. Accordingly, its expression reached to 16.5±0.25 (p=0.04) in malignant prostate tissue; 25.75±0.5 (p=0.02) in normal tissue, and 41.25±0.75 (p=0.01) in BPH tissue (FIGS. 3A-3B, 4A-4B, and 5A-5B).

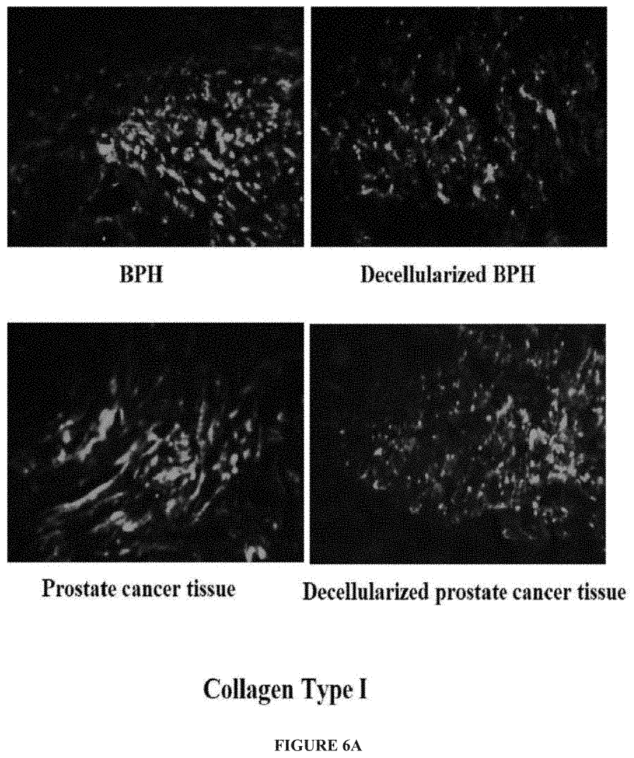

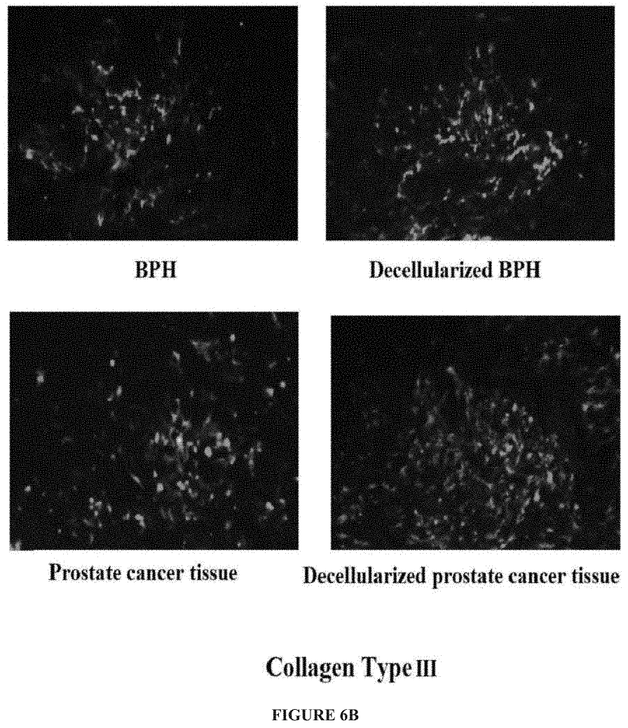

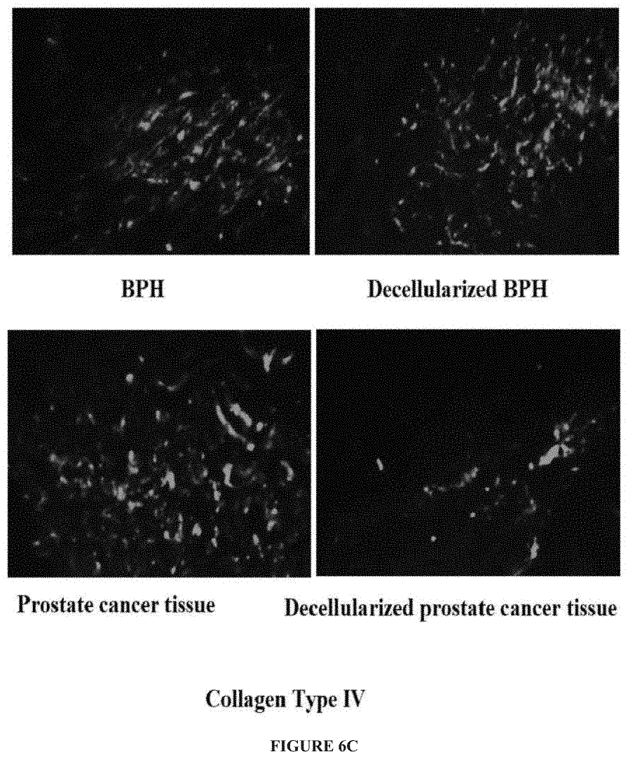

Trichrome staining of natural, BPH, and cancerous tissues demonstrated an abundance of collagen fibers before and after the decellularization; however, a slight reduction was detectable in smooth muscle fibers. Furthermore, IHC staining for type I collagen revealed approximately 60% positive reaction in normal and BPH tissues; which was also positive after decellularization process in both groups. Similar results were obtained with type III and type IV collagen (˜50% positive reaction) antibodies. IHC assessment of Type I and type III Collagen demonstrated a nearly 60% positive reaction for both markers in natural and decellularized malignant samples that was not alternated after the decellularization process. Nevertheless, a notable difference was detected between type IV collagen density of pre-decellularization malignant samples (˜60% positive reaction) and decellularized tissues (˜40% positive reaction) (FIGS. 6A-6C). The ECM ultra-structure and collagen fibers orientation was considerably preserved after the decellularization process in normal prostate samples. Nevertheless, cobblestone-shaped segments representing cellular components in SEM imaging were replaced by empty pores.

BPH specimens with coarse surfaces represented higher stromal density and homogenously thickened ECM fibers in comparison with normal prostatic tissues. No sign of disruption or structural malformation was depicted in decellularized samples of normal and BPH groups. Moreover, BPH samples contained more complex and extensive microvascular network with no notable alterations after the process. Distorted ultra-structure, disorganized ECM fibers with variable diameters, and a coarse surface were observed in SEM assessment of malignant tissues.

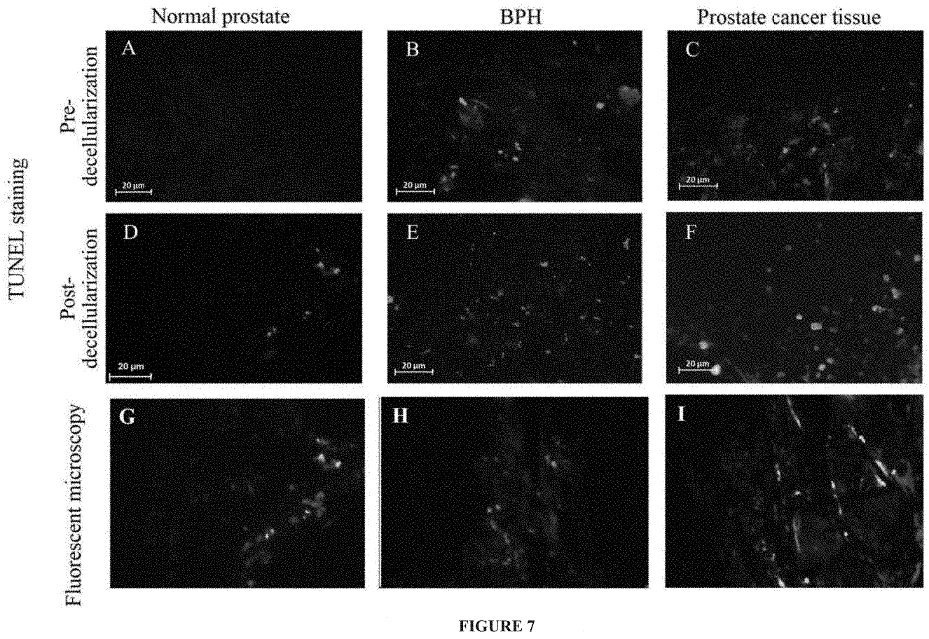

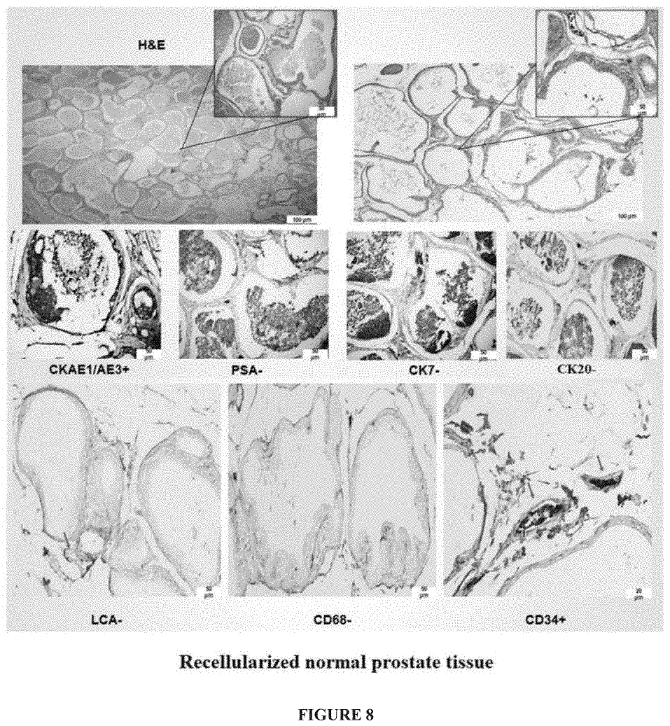

TUNEL staining was performed to distinguish double-strand DNA fragments released by apoptotic cells and to illustrate excessive DNA breakage before and after the decellularization process. Cellular malignant and non-malignant samples demonstrated condensed positive-stained concentrations predominantly in central areas. In contrast, scattered infiltration of stained DNA fragments was observed in the peripheral zone of decellularized specimens; suggestive of an enhanced apoptosis rate following the process (FIG. 7).

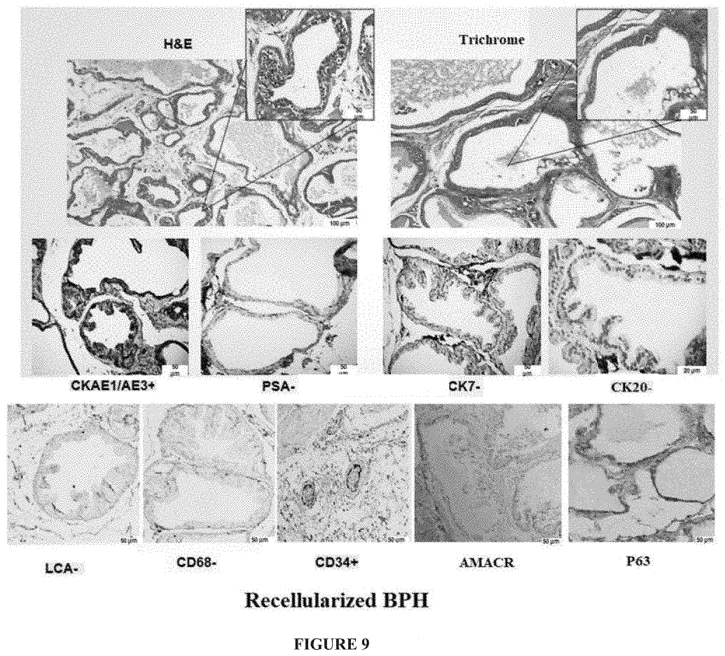

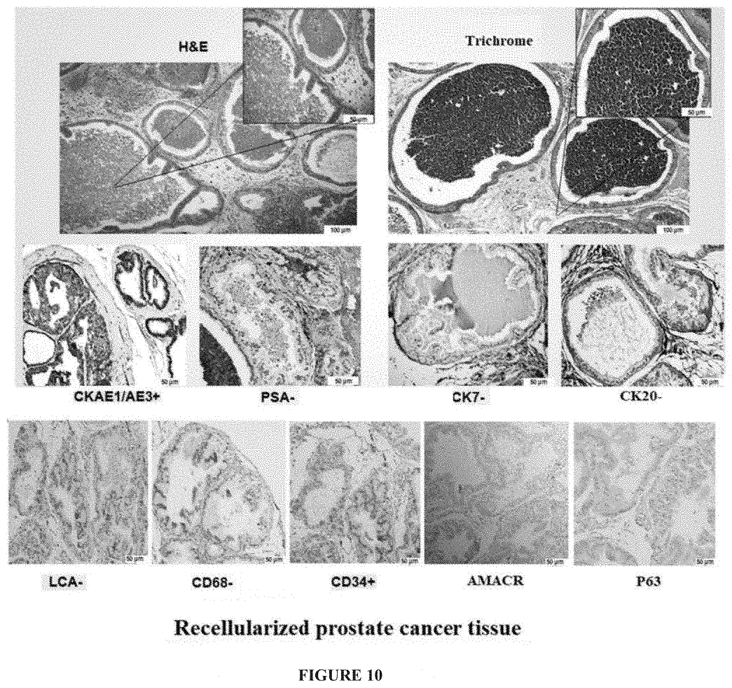

The implanted specimens were harvested six months after the surgery for histological and IHC studies. DAPI staining of bio-scaffolds after six months of follow-up endorse the presence of nuclear components, predominantly in peripheral areas. These findings were interpreted as successful in-vivo recellularization of scaffolds. Fluorescent microscopy of the samples revealed the presence of GFP-positive cells in all groups confirming host migrating cells within the recellularized scaffold in the follow-up (FIG. 7). Glandular epithelial layer was regenerated in all implanted specimens as illustrated by H&E staining. The recellularized tissues contained scattered infiltration of inflammatory cells, mostly distinguishable in peripheral areas, which was more significant in malignant specimens (p<0.05). All of the samples contained an abundance of newly-formed vessels suggestive of prominent angiogenesis within the implanted scaffolds. The concentration of stromal fibers was dominantly reduced in central areas of all groups. The samples in malignant group demonstrated the highest decrease in expression of all ECM components. Furthermore, malignant scaffolds contained enlarged glandular structures with variable size and morphology. Nonetheless, no signs of malignancy or tissue invasion were detectable in BPH and malignant samples in histological assessments. Immunostaining of harvested BPH and malignant scaffolds were negative for AMACR marker revealing the absence of cancerous cells in the recellularized tissues (FIGS. 8-10). The expression of IHC markers after implantation in different groups is depicted in Table 2.

### CONCLUSION

The outcomes illustrated complete removal of malignant and non-malignant cells in all decellularized samples. The expression of laminin and vimentin was also down-regulated after decellularization process in normal, BPH, and malignant tissues. Furthermore, malignant samples revealed prominent decreases in type IV Collagen content of the ECM. All of the implanted samples also contained an abundance of newly-formed vessels suggestive of prominent angiogenesis.

The results highlighted the potential of this study for cell-ECM interactions. Various ECM proteins such as integrins, collagen, fibronectin, and vimentin engage in cell adhesion, invasion and metastasis, which are critical for cancer progression. Hence targeting ECM is a prospective approach for further cancer therapy in patients.

1. A method of producing a decellularized tissue or organ scaffold comprising:
a) providing a tissue or organ sample, and
b) treating said tissue or organ sample with a detergent,
thereby producing a decellularized tissue or organ scaffold.

2. (canceled)

3. The method of claim 1, wherein:
i. wherein said tissue sample is a prostate tissue sample and said decellularized tissue scaffold is a decellularized prostate tissue scaffold,
ii. wherein said tissue sample is a prostate tissue sample and said decellularized tissue scaffold is a decellularized prostate tissue scaffold, wherein said prostate tissue sample is from a human cadaver or is harvested from a human subject,
iii. said detergent is a cationic detergent,
iv. said detergent is selected from SDS, Triton X-100, Trypsin, or any combinations thereof,
v. said detergent is a combination of 1% (v/v) Triton X-100, 1% sodium dodecyl sulfate (SDS), and 0.05% Trypsin solution,
vi. said treating is done under continuous shaking,
vii. said treating is done under continuous shaking performed with a mechanical shaker,
viii said treating is done under continuous shaking performed with a mechanical shaker at a speed of 70 rpm,
ix. the decellularized tissue scaffold is preserved in an antibiotic solution.

4. (canceled)

5. (canceled)

6. (canceled)

7. (canceled)

8. (canceled)

9. (canceled)

10. (canceled)

11. (canceled)

12. (canceled)

13. The method of claim 3, wherein
i. said antibiotic solution is distilled water containing 1% antibiotic/mycotic,
ii. said preserving is performed in an autoclave,
iii. said preserving prevents microbial contamination of the decellularized tissue scaffold.

14. (canceled)

15. (canceled)

16. The method of claim 1, further comprising preventing microbial contamination of the decellularized prostate tissue scaffold or further comprising sterilizing the decellularized tissue scaffold.

17. (canceled)

18. (canceled)

19. A method of producing a decellularized prostate tissue scaffold comprising:
(i) rinsing a prostate tissue sample with 1% Triton X-100;
(ii) washing said sample with phosphate buffered saline (PBS); and
(iii) preserving said sample in 1% sodium dodecyl sulfate (SDS);
thereby producing a decellularized prostate tissue scaffold.

20. The method of claim 19, wherein
a. said step (i) is performed at 37° C. for 24 hours;
b. said step (ii) is performed at 4° C. for 24 hours,
c. said step (iii) is performed at 37° C. for 48 hours,
d. said steps (i) through (iii) are performed under continuous shaking,
e. said steps (i) through (iii) are performed sequentially.

21. (canceled)

22. (canceled)

23. (canceled)

24. (canceled)

25. The method of claim 20 further comprising, following step (iii):
(iv) preserving said sample in 0.05% Trypsin solution; and
(v) rinsing said sample with PBS.

26. The method of claim 25, wherein
a. said step (iv) is performed at 37° C. for 30 minutes,
b. wherein said step (v) is performed at 4° C. for 38 hours,
c. said steps (i) through (v) are performed under continuous shaking,
d. said steps (i) through (v) are performed sequentially,
e. or any combination thereof.

27. (canceled)

28. (canceled)

29. (canceled)

30. The method of claim 25 further comprising, following step (v):
(vi) using 2% SDS solution on said sample; and
(vii) washing said sample with PBS.

31. The method of claim 30, wherein
a. said step (vi) is performed at 37° C. for 48 hours,
b. said step (vii) is performed at 4° C. for 48 hours,
c. said step (vii) is performed to remove the remaining detergent agents,
d. said steps (i) through (vii) are performed sequentially,
e. said steps (i) through (vii) are performed under continuous shaking,
f. said continuous shaking is performed with a mechanical shaker,
g. said continuous shaking is performed with a mechanical shaker regulated to circulate at 70 rpm,
h. or any combination thereof.

32. (canceled)

33. (canceled)

34. (canceled)

35. (canceled)

36. (canceled)

37. (canceled)

38. (canceled)

39. The method of claim 30 further comprising, following step (vii):
(viii) preserving the decellularized prostate tissue scaffold in an antibiotic solution.

40. The method of claim 38, wherein
a. said antibiotic solution comprises penicillin, gentamycin, and ceftriaxone,
b. said preserving is performed at 4° C.,
c. said decellularized prostate tissue scaffold is stored at 4° C.,
d. or any combination thereof.

41. (canceled)

42. (canceled)

43. The method of claim 19, wherein
a. said prostate tissue sample is provided in a phosphate buffered saline (PBS),
b. said prostate tissue sample is washed in a phosphate buffered saline (PBS) prior to decellularizing,
c. said prostate tissue sample is washed in a phosphate buffered saline (PBS) for 48 hours prior to decellularizing,
d. said prostate tissue sample is provided in a phosphate buffered saline (PBS), is sliced, and is washed with PBS for 48 hours,
e. said prostate tissue sample is from a human cadaver or is harvested from a human subject,
f. or any combination thereof.

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. The method of claim 19, wherein
a. said decellularized prostate tissue or said decellularized tissue scaffold is cell-free,
b. said tissue sample or prostate tissue sample is from a human source,
c. said tissue sample or prostate tissue sample is biologically natural,
d. said tissue sample or prostate tissue sample is normal,
e. said prostate tissue sample is from an enlarged prostate gland,
f. said prostate tissue sample is from an enlarged prostate gland due to benign prostatic hyperplasia (BPH),
g. said prostate tissue sample is a Gleason grade 2, 3, 4, or 5 cancer sample,
h. said prostate tissue sample is malignant,
i. said prostate tissue sample is malignant and reveal prominent decreases in type IV Collagen content of the decellularized prostate tissue scaffold, or
j. said decellularized prostate tissue scaffold has a decrease in laminin and vimentin compared to said prostate tissue sample.

57. (canceled)

58. (canceled)

59. (canceled)

60. (canceled)

61. (canceled)

62. (canceled)

63. (canceled)

64. (canceled)

65. (canceled)

66. The method of claim 19, further comprising repopulating the decellularized prostate tissue scaffold with cells to produce a recellularized prostate tissue scaffold.

67. The method of claim 66, wherein
a. said recellularization occurs in-vitro,
b. said recellularization occurs in-vivo,
c. said recellularization is done by in-vivo implantation of the decellularized prostate tissue scaffold in a human subject,
d. said recellularization is done by in-vivo implantation of the decellularized prostate tissue scaffold in a human subject and induces cell proliferation or cell vascularization,
e. following recellularization of the prostate tissue scaffold, the recellularized prostate tissue does not contain cancerous cells, or
f. following recellularization of the prostate tissue scaffold, the recellularized prostate tissue does not contain cancerous cells and the absence of cancerous cells in the recellularized tissues is confirmed by immunostaining of the prostate tissue scaffolds that are negative for AMACR marker.

68. (canceled)

69. (canceled)

70. (canceled)

71. (canceled)

72. (canceled)

73. (canceled)

74. A decellularized prostate tissue scaffold or an extracellular matrix comprising the decellularized prostate tissue scaffold produced by the methods of any of claim 19.

75. (canceled)

76. A recellularized prostate tissue scaffold produced by the methods of claim 66.

77. A method of treating a disorder of the prostate by implanting the decellularized prostate tissue scaffolds produced by the methods of claim 19 in a human subject.

78. The method of claim 77, wherein said disorder of the prostate is benign prostatic hyperplasia (BPH) or prostate cancer.

79. (canceled)
